(19) United States
(12) Patent Application Publication
(10) Pub. No.: US 2015/0265661 A1
(43) Pub. Date: Sep. 24, 2015

(54) PREBIOTIC EFFECT OF SIALYLLACTOSE

(71) Applicant: Trustees of Boston College, Chestnut Hill, MA (US)

(72) Inventors: David S. Newburg, Newtonville, MA (US); Zhuoteng Yu, Boston, MA (US)

(73) Assignee: Trustees of Boston College, Chestnut Hill, MA (US)

(21) Appl. No.: 14/391,739

(22) PCT Filed: Mar. 13, 2013
(86) PCT No.: PCT/US13/30764

§ 371 Date: Oct. 10, 2014

Related U.S. Application Data
(60) Provisional application No. 61/623,868, filed on Apr. 13, 2012.

(51) Int. Cl. A61K 35/745; A23L 1/29; A23L 1/30; A61K 31/717; A61K 31/702

(52) U.S. Cl. CPC A61K 35/745; A61K 31/717; A61K 31/702; A23L 1/30; A23L 1/3014; A23L 1/293; A23Y 2300/55; A23Y 2300/45; A23V 2002/00

(57) ABSTRACT

Provided herein are prebiotic compositions comprising a combination of oligosaccharides such as sialyated oligosaccharides and fusocylated oligosaccharides, and uses thereof in stimulating the proliferation of beneficial intestinal micro biota, for example, of bifidobacteria, and/or in decreasing the abundance of enteric pathogens. The prebiotic compositions can further contain a probiotic, which can be a population of bifidobacteria, lactobacilli, Bacteriodes fragilis, Bacteriodes thetaiotaomicron, Enterococcus faecalis (pro biotic strains thereof), Staphylococcus epidermides, Enterobacter aerogenes, Enterobacter cloacae, or related bacteria having similar functions.

### RELATED APPLICATION

This PCT application claims the priority to U.S. Provisional Application No. 61/623,868, filed Apr. 13, 2012, the entire content of which is herein incorporated by reference.

### BACKGROUND OF THE INVENTION

The mammalian digestive tract is typically colonized by microorganisms, also termed microbiota, including both beneficial and pathogenic microorganisms. Bacteria make up most of the mammalian gut microbiota community. For example, up to 60% of the dry mass of human feces is comprised of bacteria. It is believed that the human gut is colonized by hundreds of different species of microorganisms, with a few species typically dominating the intestinal microbiota populations.

It has been suggested that the relationship between gut microbiota and host organism is not merely a co-existence, but rather a symbiotic relationship. That is, the beneficial microorganisms (e.g., bifidofacteria) in the gut microbiota perform metabolic functions beneficial to the host, such as fermenting undigested, or non-digestible, energy substrates, modulating the host immune system, preventing growth of pathogenic bacteria, and producing nutrients that can be taken up by the host (e.g., biotin and vitamin K). An imbalance in the gut microbiota, for example, an underrepresentation of beneficial microorganisms or an overabundance of pathogenic microorganisms, can lead to disease in the host.

### SUMMARY OF THE INVENTION

The present disclosure is based on the discovery that certain combinations of sialyllactose and/or fucosylated oligosaccharides exhibited unexpectedly high prebiotic effects. For example, these combinations promoted the growth of intestinal beneficial bacteria such as bifidobacteria, particularly a number of specific bifidobacterial strains found in the digestive tract, and decreased the pH during fermentation.

Accordingly, described herein are prebiotic compositions that comprise combinations of sialylated oligosaccharide (e.g., sialyllactose) and fucosylated oligosaccharide and uses thereof in promoting the growth of beneficial bacteria such as bifidobacteria and/or inhibiting the growth of pathogenic microorganisms.

In one aspect, the present disclosure provides a prebiotic composition consisting essentially of at least one sialyllactose and at least one fucosylated oligosaccharide. The sialyllactose can be 3′-sialyllactose (3′-SL), 6′-sialyllactose (6′-SL), or a mixture thereof (e.g., at a ratio ranging from 4:1 to 1:2), and the fucosylated oligosaccharide can comprise an α1,2-fucosyl, an α1,3-fucosyl, and/or an α1,4-fucosyl residue. In some embodiments, the fucosylated oligosaccharide is a fucosylated neutral oligosaccharide. Examples of the fucosylated oligosaccharide include, but are not limited to, 2′-fucosyllactose (2-FL), 3-fucosyllactose (3-FL), lactodifucotetraose (LDFT), or a mixture thereof (e.g., a mixture of: 2′-FL and 3-FL; a mixture of 2′-FL and LDFT; a mixture of 3-FL and LDFT; or a mixture of 2′-FL, 3-FL, and LDFT). In one example, the composition consists essentially of a mixture of 3′-SL, 6′-SL, 2′-FL, 3-FL, and LDFT.

If desired, any of the prebiotic compositions described above can further contain a probiotic, which can be a population of bifidobacteria, lactobacilli, Bacteriodes fragilis, Bacteriodes thetaiotaomicron, Enterococcus faecalis (probiotic strains thereof), Staphylococcus epidermides, Enterobacter aerogenes, Enterobacter cloacae, or related bacteria having similar functions. In some embodiments, the population of bifidobacteria is B. longum (e.g., B. longum JCM7007, JCM7009, JCM7010, JCM7011, JCM1210, JCM1260, JCM1272, JCM11347, or ATCC15708), B. infantis (e.g., B. infantis ATCC15697), or a mixture thereof.

In another aspect, the present disclosure provides a method of increasing the proliferation of a beneficial bacterium, e.g., bifidobacteria, with a prebiotic composition comprising a sialyllactose and a fucosylated oligosaccharide (e.g., those described above) in an amount effective in increasing the proliferation of the population of the beneficial bacterium (e.g., a bifidobacteria population). The sialyllactose can be 3′-sialyllactose (3′-SL), 6′-sialyllactose (6′-SL), or a mixture thereof. The fucosylated oligosaccharide can comprise an α1,2-fucosyl, an α1,3-fucosyl, and/or an α1,4-fucosyl residue. In some embodiments, the fucosylated oligosaccharide is a fucosylated neutral oligosaccharide. Examples of fucosylated oligosaccharide include, but are not limited to, 2′-fucosyllactose (2-FL), 3-fucosyllactose (3-FL), lactodifucotetraose (LDFT), or a mixture thereof (e.g., those described above).

The just-described method can be performed either in vitro or in vivo. For example, the contacting step can be performed by administering any of the prebiotic compositions described herein to a subject in need of the treatment. In some embodiments, the prebiotic composition is administered orally to the subject.

A subject who needs to be treated by a prebiotic composition as described herein can be a human (e.g., a human infant such as a neonatal infant). In some embodiments, the subject (e.g., a human) is suffering from, suspected of having, or at risk for a disease associated with an underrepresentation of beneficial microorganisms or the presence or overabundance of pathogenic bacteria in the intestine. In other embodiments, the subject is suffering from, suspected of having, or at risk for irritable bowel syndrome or inflammatory bowel disease.

If necessary, the prebiotic composition can be administered to a subject as described herein in an amount effective to decrease the pH in the microenvironment of the beneficial bacterium (e.g., a bifidobacteria population such as Bifidobacterium longum, Bifidobacterium infantis, or a mixture thereof). Alternatively, the prebiotic composition can be administered to the subject in an amount effective to decrease the proliferation rate of a pathogenic bacterium (e.g., Escherichia coli or Clostridium perfringens). The prebiotic compositions to be used in the methods described herein can further comprise a probiotic, e.g., bifidobacteria such as B. longum, B. infantis, or a mixture thereof. In some embodiments, the population of bifidobacteria is B. longum JCM7007, JCM7009, JCM7010, JCM7011, JCM1210, JCM1260, JCM1272, JCM11347, ATCC15708, B. infantis ATCC15697, or a mixture thereof.

Also within the scope of this disclosure are (i) pharmaceutical compositions for use in promoting the growth of beneficial bacteria in a subject, for treating a disease associated with an underrepresentation of beneficial microorganisms or the presence or overabundance of pathogenic bacteria in the intestine, or for treating irritable bowel syndrome or inflammatory bowel disease, and (ii) use of the pharmaceutical compositions for the manufacture of medicaments for the treatment of the diseases noted above. The pharmaceutical compositions can comprise any of the prebiotic compositions described herein and a pharmaceutically acceptable carrier. In some examples, the prebiotic compositions comprise a combination of sialylated (e.g., sialyllactose) and fucosylated oligosaccharides as described herein and one or more probiotics.

The details of certain exemplary, non-limiting embodiments of the invention are set forth in the description below. Other features or advantages of the present invention will be apparent from the following drawings and detailed description of several examples, and also from the appended claims.

### BRIEF DESCRIPTION OF THE DRAWINGS

FIG. 1 is a diagram showing the variation of pH (panel A) and lactate concentration (panel B) in the fermentation culture media in the presence or absence of human milk oligosaccharides (HMOS) or Fructo-oligosaccharides (FOS).

FIG. 2 is a diagram showing microbiota distribution in faecal samples in the presence of HMOS and FOS. The number of different bacteria species in the fermentation culture supplement with or without HMOS as determined by qPCR. Panel A: bifidobacteria. Panel B: Clostridium perfringens. Panel C: E. coli.

FIG. 3 is a diagram showing human milk oligosaccharide consumption profiles of different donor faecal microbiota, as determined using LC-MS.

FIG. 4 is a bar graph showing growth increase (panel A) and pH decrease (panel B) percentages of different bacteria with fucosylated oligosaccharides 2′-FL, 3-FL, LDFT, HMOS and the fructooligosaccharide positive control, FOS.

FIG. 5 is a bar graph showing growth increase (panel A) and pH decrease (panel B) percentages of different bacteria as indicated with fucosylated oligosaccharide 2′-FL, 3-FL, and/or LDFT; siallyllactose 3′-SL and/or 6′-SL; a combination thereof; HMOS; and FOS.

FIG. 6 is a bar graph showing growth increase (panel A) and pH decrease (panel B) percentages of different bacteria as indicated with fucosylated oligosaccharide 2′-FL, 3-FL, and/or LDFT; siallyllactose 3′-SL and/or 6′-SL; a combination thereof; HMOS; and FOS.

FIG. 7 is a graph showing structures of exemplary siallyllactoses and fucosylated oligosaccharides.

### DETAILED DESCRIPTION OF THE INVENTION

Dietary glycans that are indigestible by animals can be utilized by beneficial bacteria, thereby promoting colonization of gut microbiota, particularly colonization of beneficial bacteria, which is important for health. Gut colonization is the establishment or maintenance of a live microorganism population within the digestive tract of a host organism. It can be the colonization of the entire digestive tract as well as partial colonization, for example, of only a subsection of the gut (e.g., or the small intestines, of the large intestines, or of the stomach). In mammals, gut colonization begins at birth and breastfeeding is often associated with a typical microbiota rich in beneficial bacteria, such as Bifidobacteria. This indicates that certain components in milk possess prebiotic effects.

Beneficial bacteria or beneficial microbiota are microorganisms (e.g., bacteria, fungi, protozoa), also known as probiotics, confer beneficial effects to the host organism when colonized in the gut of a host organism. For example, they metabolize a food ingredient that is non-digestible to the host organism, modulate the host immune system in a non-pathogenic manner, prevent growth of pathogenic bacteria, and/or produce nutrients that can be taken up by the host (e.g., biotin and vitamin K). Beneficial bacteria include, but are not limited to bifidobacteria, lactobacilli, Bacteriodes fragilis, Bacteriodes thetaiotaomicron, Enterococcus faecalis (probiotic strains of E. faecalis), Staphylococcus epidermides, Enterobacter aerogenes, and Enterobacter cloacae.

Pathogenic bacteria refer to any bacteria that can cause and/or do cause a disease or condition in a subject. In some embodiments, the term includes pathogenic bacteria that colonize the gut of a subject. In some embodiments, the term includes material that are pathogenic if present or overabundant in the gut of a subject. Exemplary pathogenic bacteria include, but are not limited to Escherichia coli, Clostridium perfringens, Listeria monocytogenes, Listeria innocua, Staphylococcus aureus, Enterococcus faecalis (virulent strains of E. faecalis), and Enterococcus faecium.

The present disclosure is, at least partially, based on the discovery that human milk oligosaccharides (HMOS), particularly the fucosylated oligosaccharides and siallyllactose described herein, can act as prebiotics that serve as a source of energy and nutrients for desired bacteria to colonize the infant intestine. Prebiotics are food ingredients, for example, oligosaccharides, that are non-digestible by a subject (e.g., by a mammal such as a human), and that stimulates the growth or activity of one or more beneficial bacteria (e.g., bifidobacteria) in the digestive system and/or inhibit the growth or activity of one or more pathogenic bacteria in the digestive system. A prebiotic may selectively stimulate the growth and/or activity of one or a limited number of bacteria in the subject's digestive tract.

Accordingly, described herein are prebiotic compositions comprising combinations of sialylated oligosaccharide (e.g., siallyllactose) and fucosylated oligosaccharides and uses thereof for promoting the growth/activity of beneficial bacteria and/or inhibiting the growth and/or activity of pathogenic bacteria.

### Prebiotic Compositions

The prebiotic compositions described herein comprise a combination of oligosaccharides (e.g., those found in milk such as sialylated oligosaccharides and fucosylated oligosaccharides) that possess prebiotic activities. These compositions are therefore useful (either in vivo or in vitro), for example, to promote one or more beneficial gut bacteria (e.g., bifidobacteria, including the specific strains disclosed in the Example below) in a subject, which can be a human such as a human neonatal infant, and to inhibit the growth of one or more pathogenic bacteria.

In some embodiments, a prebiotic composition described herein consists essentially of at least one sialyated oligosaccharide (e.g., sialyllactose) and at least one fucosylated oligosaccharide. Such a prebiotic composition comprises the specified active ingredients, e.g., the specified oligosaccharides, as well as other agents that do not materially affect the basic and novel characteristics of the composition. The specified active ingredients can be the major prebiotic agents in the composition. In some examples, the specified active ingredients (e.g., the specified oligosaccharide prebiotics) constitute at least about 25%, 30%, 35%, 40%, 50%, at 60%, 70%, 80%, 90%, or 95% of the respective composition by weight. In other examples, the specified oligosaccharide prebiotics constitute at least about 50%, 60%, 70%, 80%, 90%, or 95% of the total sugar content in the composition by weight. In one example, the specified oligosaccharides are the only prebiotics in the composition.

Oligosaccharides are polymeric molecules comprising two or more saccharide monomers. An oligosaccharide can contain 2, 3, 4, 5, 6, 7, 8, 9, 10, or more saccharides monomers.

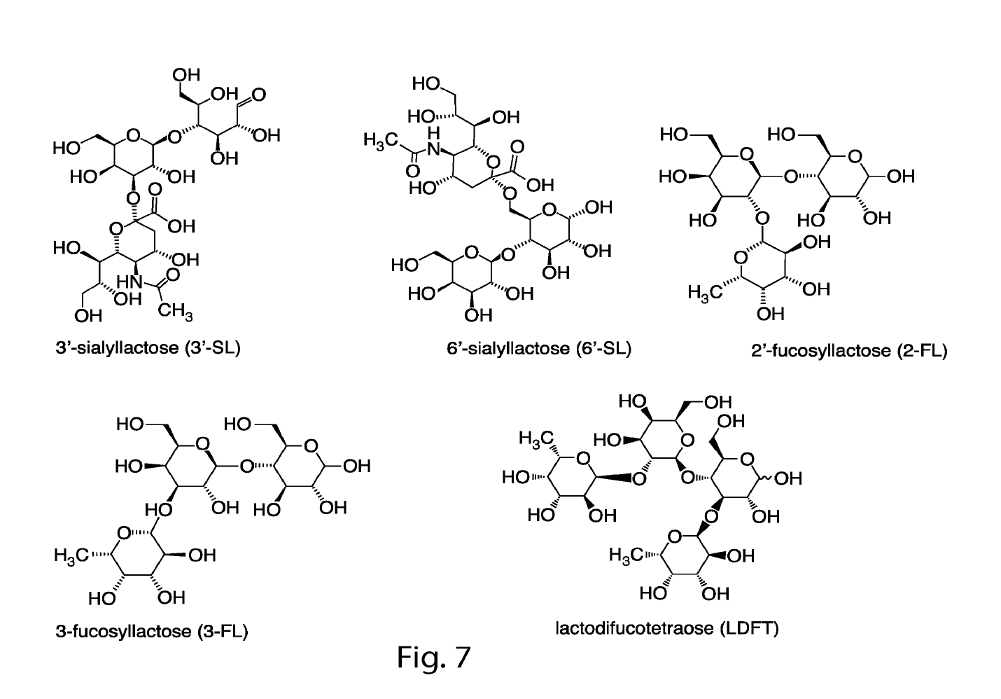

Sialyated oligosaccharides are oligosaccharide molecules containing one or more sialic acid moieties. Exemplary sialyated oligosaccharides include 3′-SL and 6′SL. See FIG. 7. The prebiotic composition can contain 3′-sialyllactose (3′-SL), 6′-sialyllactose (6′-SL), or both. When the prebiotic composition described herein contains a mixture of 3′-SL and 6′-SL, the ratio between 3′-SL and 6′SL (e.g., weight/weight, volume/volume, mol/mol) can be within the range of 10:1-1:10, e.g., 5:1-1:5, 4:1-1:2, or 2:1-1:2. In some examples, the ratio between 3′-SL and 6′-SL is about 1:1, about 1:2, about 1:3, about 1:4, about 1:5, about 1:6, about 1:7, about 1:8, about 1:9, about 1:10, about 1:10, about 1:20, about 1:30, about 1:40, about 1:50, about 1:60, about 1:70, about 1:80, about 1:90, about 1:100, about 2:1, about 3:1, about 4:1, about 5:1, about 6:1, about 7:1, about 8:1, about 9:1, about 10:1, about 20:1, about 30:1, about 40:1, about 50:1, about 60:1, about 70:1, about 80:1, about 90:1, or about 100:1. In other examples, the ratio between 3′-SL and 6′-SL can be within the range of 1000:1-1:1000, e.g., 100:1-10:1, 10:1-1:1, 10:1-5:1, 1:100-1:10, 1:10-1:1, 1:10-1:5.

Fucosylated oligosaccharides are oligosaccharide molecules that comprise one or more fucose monomers, which can be in α1,2-, α1,3-, or α1,4-linkage. In some examples, the fucosylated oligosaccharides contained in the prebiotic compositions described herein are neutral oligosaccharides, which do not contain acidic or basic moieties at physiologic pH. Such fucosylated oligosaccharides include, but are not limited to, 2′-fucosyllactose (2-FL), 3-fucosyllactose (3-FL), and lactodifucotetraose (LDFT). See FIG. 7. The prebiotic compositions described herein can contain 2′-FL, 3-FL, LDFT, or any combination thereof. In one example, the composition contains 3′-SL, 6′-SL, 2′-FL, 3-FL, and LDFT.

In some embodiments, the prebiotic composition contains sialyated oligosaccharide(s) and fucosylated oligosaccharide(s) at a ratio ranging from 100:1-1:100, e.g., 20:1-1:20, 10:1-1:10, 5:1-1:5, 4:1-1:2, or 2:1-1:2.

A prebiotic composition described herein may contain one or more prebiotic oligosaccharide (e.g., a sialylated oligosaccharide such as 3′-SL or 6′-SL; or a fucosylated oligosaccharide such as 2′-FL, 3-FL, or LDFT), which constitute about 0.2-98% (e.g., at least 0.5%, 1%, 5%, 10%, 20%, 30%, 50%, 75%, or 80%) of the total composition (e.g., as weight of solid ingredients per weight of dry matter, or per volume of solvent in the case of a liquid formulation).

When necessary, any of the prebiotic compositions described herein can further comprise a probiotic (e.g., bifidobacteria, also referred to as lactobacillus bifidus), i.e., a population of live microorganisms, which, when administered in adequate amounts, confer a health benefit to the host. Probiotic bifidobacteria are well known to those of skill in the art, and exemplary bifidobacteria useful according to aspects of this invention include, but are not limited to, B. longum and B. infantis (see, e.g., Schell et al., The genome sequence of Bifidobacterium longum reflects its adaptation to the human gastrointestinal tract. Proc Natl Acad Sci USA 2002, 99 (22):14422-7; Fukuda et al., Bifidobacteria can protect from enteropathogenic infection through production of acetate. Nature 2011, 469, 543-547; and Whorwell et al., Efficacy of an encapsulated probiotic Bifidobacterium infantis 35624 in women with irritable bowel syndrome. American Journal of Gastroenterology 2006, July; 101(7):1581-90, the entire contents of each of which are incorporated by reference herein). Exemplary bifidobacteria strains useful according to some aspects of this invention include, but are not limited to B. longum strains JCM7007, JCM7009, JCM7010, JCM7011, JCM1210, JCM1260, JCM1272, JCM11347, and ATCC15708, and B. infantis strain ATCC15697. Additional probiotics useful in accordance with aspects of this invention include, but are not limited to, Bacillus coagulans, Lactobacillus acidophilus, Lactobacillus paracasei, Lactobacillus johnsonii, Lactobacillus plantarum, Lactobacillus reuteri, Saccharomyces boulardii, Lactobacillus rhamnosus, and Lactobacillus plantarum.

In some embodiments, the composition comprises a single probiotic, for example, a single species or strain of probiotic bacteria as described herein. In other embodiments, the composition comprises a plurality of probiotics, for example, a mixture of two or more probiotic strains or species described herein or known to those of skill in the art.

In some embodiments, the probiotic is comprised in the composition in the form of live, microorganisms. In some embodiments, the probiotic is comprised in the composition in the form of actively growing and/or dividing microorganisms. In some embodiments, the probiotic is comprised in the composition in a dormant form, such as a spore or an endospore.

### Methods of Using Prebiotic Compositions for Promoting Growth of Beneficial Bacteria

Given the prebiotic activity of any of the compositions described herein, such compositions can be used to promote the growth and/or activity of one or more beneficial bacteria either in vitro or in vivo. Such compositions can also be used to inhibit the growth and/or activity of one or more pathogenic bacteria in vitro or in vivo.

In some embodiments, the methods described herein comprise contacting beneficial microbiota (e.g., a population of bifidobacteria) with an effective amount of any of the prebiotic compositions described herein to increase the proliferation of beneficial microbiota. The contacting step can be performed in vitro, e.g., in a culture dish. Alternatively, this step can be performed in vivo, e.g., by administering the prebiotic to a subject in need of the treatment.

The term “increasing the proliferation of,” as used herein in the context of microbiota or microorganisms, for example, of beneficial gut microorganisms, such as bifidobacteria, refers to increasing the rate of cell proliferation and/or cell survival of the respective microbiota or microorganisms. For example, a prebiotic composition that increases the proliferation of microorganisms may be a composition that, when contacted with a population of the microorganism, results in an increase in cell division rates and/or survival rates among the microorganisms by at least 20%, 40%, 60%, 80%, 1-fold, 2-fold, 5-fold, 10-fold, 50-fold, 100-fold, or 200-fold, as compared to the rates in the absence of the composition.

Similarly, the term “decreasing the proliferation of” or “inhibiting the growth of” as used herein in the context of pathogenic microorganisms, for example, of pathogenic gut bacteria, refers to decreasing the rate of cell proliferation and/or cell survival of the respective microorganisms. For example, a prebiotic composition that increases the proliferation of beneficial microorganisms may also be a composition that decreases the proliferation of pathogenic microorganisms by at least 20%, 40%, 50%, 60%, 70%, 80%, 90%, or 95%, as compared to the rates in the absence of the composition. The composition itself may exert a direct anti-proliferative effect on the pathogenic microorganisms. Alternatively, the anti-proliferative effect may be a secondary effect of the composition. An exemplary secondary effect could be that uptake and metabolization of the composition by beneficiary got microbiota results in a change in the gut microenvironment that is not favorable for the growth and proliferation of pathogenic microorganisms. For example, in some embodiments, uptake and metabolization of a prebiotic composition described herein by beneficial gut microbiota, for example, by bifidobacteria, results in a shift in the pH of the microenvironment of the microbiota towards an acidic pH, which is unfavorable to the proliferation and/or survival of certain pathogenic bacteria.

In one example, a prebiotic composition is administered to a subject in need thereof in an amount effective to increase the proliferation of a beneficial microorganism in the subject's intestine by at least about 25%, at least about 50%, at least about 75%, at least about 1 fold, at least about 2 folds, at least about 3 folds, or at least about 5 folds. The amount of the composition can be effective in promoting the growth and/or activity of one or more beneficial bacteria, e.g., a population of bifidobacteria, lactobacilli, Bacteriodes fragilis, Bacteriodes thetaiotaomicron, Enterococcus faecalis (probiotic strains of E. faecalis), Staphylococcus epidermides, Enterobacter aerogenes, or Enterobacter cloacae. In some embodiments, the population of bifidobacteria is B. longum (e.g., B. longum JCM7007, JCM7009, JCM7010, JCM7011, JCM1210, JCM1260, JCM1272, JCM11347, or ATCC15708), B. infantis (e.g., B. infantis ATCC15697), or a mixture thereof.

Alternatively, the prebiotic composition can be administered to the subject in an amount effective to decrease the proliferation rate of a pathogenic bacterium (e.g., Escherichia coli or Clostridium perfringens). In some embodiments, the prebiotic composition is administered to the subject in an amount effective to decrease the proliferation rate of the pathogenic bacterium by at least about 25%, at least about 30%, at least about 40%, at least about 50%, at least about 60%, at least about 70%, at least about 80%, at least about 90%, at least about 95%, at least about 98%, or at least about 99%.

The method described herein can further comprise monitoring the proliferation of the beneficial microorganism in the subject, for example, by fecal examinations. Methods for monitoring microbiota and assessing the proliferation of specific beneficial microorganisms in a subject's intestine are well known to those of skill in the art, and exemplary, non-limiting methods are described in Moro et al, Dosage-related bifidogenic effects of Galacto- and fructooligosaccharides in formula fed term infants. Journal of pediatric Gastroenterology and Nutrition 34:291-295 2002, and Campbell et al., Selected indigestible oligosaccharides affect large bowel mass, cecal and fecal short-chain fatty acids, pH and microflora in rats. J. Nutr 127:130-136 1997; the entire contents of both of which are incorporated herein by reference. Additional methods suitable for monitoring the effect of a prebiotic composition on a subject's microbiota according to some aspects of this invention are known or will be apparent to those of skill in the art, and the invention is not limited in this respect.

In still another example, a prebiotic composition described herein is administered to a subject in an amount effective to decrease the pH in the microenvironment of the bifidobacteria. Microenvironment can be a small or relatively small habitat or environment, e.g., a subject's intestine or a part thereof. A microenvironment can be, at least partially, isolated from surrounding environments, for example, by a physical barrier. In some embodiments, a microenvironment embraces a bacterial cell or cell population and its immediate surroundings, which can immediately affected by the presence of the bacterial cell or cells or the metabolism of the cells.

Methods of determining the proliferation rate of beneficial intestinal microorganisms, pathogenic bacteria, and the pH of a microenvironment, for example, within the intestinal substrate of a subject, are well known to those of skill in the art. Such methods typically include assessing the intestinal microbiota, or the intestinal substrate of the subject before restriction of the prebiotic composition is commenced, and monitoring the intestinal microbiota, or the intestinal substrate of the subject during and/or after administration of the prebiotic composition. Some such methods are described herein, and additional methods will be apparent to the skilled artisan. For a description of some exemplary, nonlimiting methods see, e.g., Moro et al, Dosage-related bifidogenic effects of Galacto- and fructooligosaccharides in formula fed term infants. Journal of pediatric Gastroenterology and Nutrition 34:291-295 2002, and Campbell et al., Selected indigestible oligosaccharides affect large bowel mass, cecal and fecal short-chain fatty acids, pH and microflora in rats. J. Nutr 127:130-136 1997; the entire contents of both of which are incorporated herein by reference. It will be appreciated that the present invention is not limited in this respect.

In another example, a prebiotic composition is administered to a subject in an amount effective to increase the abundance of a beneficial microorganism, for example, of a bifidobacteria, in the intestine of the subject by at least 2-fold, at least 5-fold, at least 10-fold, at least 20-fold, at least 50-fold, at least 100-fold, at least 500-fold, at least 1000-fold, or at least 1000-fold, as compared to the abundance of the beneficial microorganism at the outset of the administration. Such a treatment can last for a suitable period of time until the desired result is achieved. Before the treatment, the subject can have no detectable level of the beneficial microorganism in his or her intestine. The subject can be administered with a prebiotic composition, which preferably contains at least one probiotic, at an amount and for a time sufficient for the beneficial microorganism to colonize the intestine of the subject.

In yet another example, a prebiotic composition is administered to a subject in an amount and for a period of time effective to decrease the abundance of a pathogenic microorganism, for example, of an enteric pathogen, such as C. perfringens, in the intestine of the subject by at least 2-fold, at least 5-fold, at least 10-fold, at least 20-fold, at least 50-fold, at least 100-fold, at least 500-fold, at least 1000-fold, or at least 1000-fold, as compared to the abundance of the pathogenic microorganism at the outset of the administration. The subject can be treated for a suitable period of time with an effective amount of the composition such that, after the treatment, the abundance of the pathogenic microorganism is below a measurable level.

A subject in need of the treatment described herein can be a subject suffering from, suspected of having, or at risk for a disease associated with an underrepresentation of beneficial microorganisms or the presence or overabundance of pathogenic bacteria in the intestine. Such a subject may exhibit a clinical symptom indicating the underrepresentation of beneficial microorganisms or the presence or overabundance of pathogenic bacteria in the subject's intestine. Alternatively, the subject may be at risk of contracting a disease associated with an underrepresentation of beneficial microorganisms or the presence or an overabundance of pathogenic bacteria in the subject's intestine. For example, in some embodiments, the subject is a subject with a history of such diseases. The underrepresentation of beneficial microorganisms or the presence or an overabundance of pathogenic bacteria and/or beneficial bacteria in the intestinal microbiota can be examined in a candidate subject to determine whether treatment is needed.

The subject can also be a subject suffering from, suspected of having, or at risk for a digestive tract disease, such as irritable bowel syndrome or inflammatory bowel disease.

The term “subject,” as used herein, refers to a mammal, for example, a primate, a non-human primate, a human, a dog, a cat, a sheep, a goat, a cattle, a horse, a pig, a mouse, a rat, a guinea pig, a domestic animal, a wild animal, a farm animal, or a laboratory animal. In some examples, the subject is an infant, e.g., a neonatal infant. In other examples, the subject is an adolescent or an adult. Other developmental stages, for example prenatal and perinatal stages are also included in some embodiments.

A subject suspected of having, or at risk for a disease refers to a subject having an elevated level of suspicion of the presence of the disease or an elevated level of risk for contracting the disease, as compared to an average level of suspicion for average risk level. For example, a subject manifesting clinical symptoms of a specific disease has an elevated level of suspicion of the presence of the disease, even in the absence of an objective clinical diagnosis. For another example, the subject may be predisposed to contracting a specific disease, for example, because of the subject's genetic makeup, or because of exposure to environmental pathogens, or because of the presence of behavioral risk factors, such as dietary or other behavioral habits.

The method described herein can further comprise assessing the subject's intestinal microbiota before administration of the prebiotic composition is commenced. Alternatively or in addition, the method can comprise monitoring the subject's intestinal microbiota during and/or after administration of the prebiotic composition. If no increase in the proliferation of a beneficial microorganism, e.g., a bifidobacteria, is detected in the subject after about a suitable period, e.g, 1 week, 2 weeks, 3 weeks, or about 1 month after administration has commenced, then the dosage of the prebiotic composition can be increased. If a beneficial change in the subject's intestinal microbiota is detected, for example, an increase in the proliferation of a beneficial microorganism, then the dosage of the prebiotic composition may be decreased or administration may be phased out altogether.

In some embodiments, a prebiotic composition described herein is administered to a subject to treat a disease or disorder in the subject, such as irritable bowel syndrome or inflammatory bowel disease, or a disease associated with the presence or an overabundance of pathogenic microorganisms in the subject's intestine (e.g., diarrhea, gastrointestinal infection, necrotizing enterocolitis, Crohn's disease, or diverticulitis). The term “treating” as used herein refers to the application or administration of a composition including one or more active agents to a subject in who has any of the diseases described herein, a symptom of the disease, or a predisposition toward the disease, with the purpose to cure, heal, alleviate, relieve, alter, remedy, ameliorate, improve, or affect the disease, the symptoms of the disease, or the predisposition toward the disease.

For example, in some embodiments, a prebiotic composition as described herein is ministered to a subject presenting with at least one clinically manifest symptom of irritable bowel syndrome or inflammatory bowel disease, and having an overabundance of a pathogenic bacterium, for example, Clostridium perfringens, in her intestine. In some embodiments, a fecal exam is performed at the outset of treatment with the prebiotic composition, and subsequent fecal exams are performed after 1 week and after 4 weeks of treatment, and cell counts of pathogenic bacteria are compared to those observed in the initial exam to monitor the effectiveness of the treatment schedule. If after one week no significant reduction in the number of Clostridium perfringens is detected, the dosage of the prebiotic composition is increased.

The method described herein can be applied to a subject who has been subjected to or is undergoing another treatment, e.g., antibiotic treatment. In some embodiments, administration of the prebiotic composition is commenced immediately after the antibiotic treatment schedule has ended. In other embodiments, administration of the prebiotic composition is commenced about one day, about two days, about three days, about four days, about 5 days, about 6 days about 1 week, about 2 weeks, about 3 weeks, or about a month after the antibiotic treatment schedule has ended. In some embodiments, a prebiotic composition is administered to a subject concurrently with an antibiotic treatment. In some such embodiments, administration is continued beyond the end point of antibiotic treatment.

In some embodiments, a method is provided in which a prebiotic composition as provided herein is administered to a subject in need of or in a stage of intestinal colonization with microbiota. Prebiotic compositions comprising a probiotic, for example, a live bifidobacterial population, are particularly suited for administration to such subjects, which include neonatal infants, and subjects who have undergone a treatment that has created an imbalance in or has eradicated most or all of the subject's microbiota, such as an oral antibiotics treatment.

### Formulations, Routes of Administration, and Dosage

The prebiotic compositions described herein may be formulated and administered in any suitable form known to those of skill in the art. For enteral administration, the prebiotic compositions of the invention may be formulated into preparations in solid, semi-solid, gel, or liquid forms such as tablets, capsules, powders, granules, solutions, depositories, gels, and injections. Compositions suitable for oral administration may be presented as discrete units, such as capsules, tablets, lozenges, each containing a predetermined amount of an active agent. Other compositions include suspensions in aqueous liquids or non-aqueous liquids such as a syrup, elixir, gels, or emulsions.

In some embodiments, a composition described herein is administered to a subject via an enteral route, for example, orally in the form of a powder, powdered drink, liquid, capsule, or pill. In some embodiments, a prebiotic composition is administered to a subject in a single dose, while in other embodiments, multiple doses are administered over a certain time period. For example, in some embodiments, a prebiotic composition as described herein is administered at 1, 2, 3, 4, or 5 doses per day, preferably at one dose per day. In some embodiments, administration is continued over a period of about 1 week, about 2 weeks, about 3 weeks, about 4 weeks, about 1 month, about 5 weeks, about 6 weeks, about 2 months, about 3 months, about 4 months, about 5 months, about 6 months, or about 1 year. In some embodiments, administration is continued until a clinical symptom in the subject is ameliorated. In some embodiments, administration is continued until a beneficial microorganism, for example, a bifidobacteria, is detected in the intestinal microbiota of the subject, or an increase in the cell numbers of the beneficial microorganism is detected in the intestinal microbiota of the subject.

It should be understood that, in some embodiments, different components of a prebiotic composition described herein are administered via the same route, for example, orally, while in other embodiments, different components of a prebiotic composition may be administered via different routes. For example, in some embodiments, prebiotic oligosaccharides may be administered via one enteral rout, while a probiotic may be administered via a different enteral route. In some embodiments, a composition as provided herein is formulated as a solid state composition, comprising one or more of the components in the form of a powder, granules, pellets, pills, or in crystalline form. In some embodiments, such a solid state composition comprises a mixture of two or more oligosaccharides in solid form. Compositions suitable for oral administration may be presented as discrete units, such as capsules, tablets, lozenges, each containing a predetermined amount of an active agent. Other compositions include suspensions in aqueous liquids or non-aqueous liquids such as a syrup, elixir or an emulsion.

For oral administration, an agent can be formulated readily by combining with pharmaceutically acceptable carriers well known in the art. Such carriers enable an agent of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

In some embodiments, a prebiotic composition comprising two or more components, e.g., two or more prebiotic oligosaccharides or a prebiotic oligosaccharide and a probiotic, as described herein, may be formulated as a combination, or mixture, of all components, e.g., a mixture of all oligosaccharides and/or probiotic(s), e.g., in the form of a solid, liquid, powder, gel, or other form described herein. In some embodiments, a prebiotic composition comprising two or more prebiotic oligosaccharides may be formulated and/or administered as individual agents or compounds separately, or as a mixture of any subcombination and on or more additional components separately. It should be appreciated that the prebiotic agents of a composition described herein may be administered at the same time, contemporaneously, or during a course of treatment. Accordingly, in some embodiments, a combined preparation of two or more prebiotic oligosaccharides described herein may be provided for simultaneous, separate, or sequential use in therapy as described herein.

In some embodiments, fixed ratios of prebiotic oligosaccharides and/or probiotics described herein, are administered, for example in a solid or liquid formulation containing all oligosaccharides and probiotics, if any, at a specific ratio or by combining individual oligosaccharides and/or probiotics to result in a certain ratio. Ratios can be based, for example, on weight, volume, and/or biologic activity of the specific prebiotic or probiotic agent, or a combination thereof.

Some aspects of this invention provide pharmaceutical compositions comprising a prebiotic and/or a probiotic agent described herein. Pharmaceutical compositions according to some aspects of this invention comprise an effective amount of a prebiotic and/or a probiotic agent as described herein, either in solid form, or dissolved or dispersed in a pharmaceutically acceptable carrier. Preferably, pharmaceutical compositions are sterile, or, where probiotics are included, comprise an isolated population of the probiotic(s) that is free of any pathogenic microorganism or inflammation-causing agents. The phrases “pharmaceutical or pharmacologically acceptable” refers to molecular entities and compositions that do not generally produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

The term “pharmaceutically-acceptable salts” in this respect refers to the relatively non-toxic, inorganic or organic acid addition salts of agents of the present invention. These salts can be prepared in situ in the administration vehicle or the dosage form manufacturing process, or by separately reacting a purified prebiotic agent of the invention with a suitable organic or inorganic acid, and isolating the salt thus formed during subsequent purification. Representative salts include the bromide, chloride, sulfate, bisulfate, phosphate, phosphonate, nitrate, acetate, valerate, oleate, palmitate, stearate, laurate, benzoate, lactate, tosylate, citrate, maleate, fumarate, succinate, tartrate, napthylate, mesylate, glucoheptonate, lactobionate, and laurylsulphonate salts and the like. See, for example, Berge et al. (1977) J. Pharm. Sci. 66:1-19.

In other cases, the agents of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al. (1977) J. Pharm. Sci. 66:1-19.

In embodiments where a prebiotic composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof. In many cases, it will be advisable to include an isotonic agent, such as, for example, sugars, sodium chloride or combinations thereof. In some embodiments, the formulations of the invention are administered in pharmaceutically acceptable liquid solutions, which may routinely contain pharmaceutically acceptable concentrations of salt, buffering agents, preservatives, compatible carriers, adjuvants, and, optionally, other therapeutic ingredients.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. A composition may comprise different types of carriers depending on whether it is to be administered in solid, liquid or aerosol form, and whether it need to be sterile.

In certain embodiments, pharmaceutical compositions may comprise, for example, at least about 0.1% of a prebiotic and/or probiotic compound, or a mixture of such compounds. In other embodiments, the prebiotic and/or probiotic compound, or the mixture of such compounds may comprise between about 2% to about 75% in weight/weight or weight/volume of the composition, or between about 25% to about 60%, or up to 99% of the composition, for example, and any range derivable therein.

The prebiotic agents of the invention may be derivatized in various ways. As used herein “derivatives” of the agents provided herein include salts (e.g., pharmaceutically acceptable salts), complexes, esters, such as in vivo hydrolysable esters, free acids or bases, polymorphic forms of the compounds, solvates (e.g., hydrates), prodrugs, coupling partners and protecting groups. By “prodrugs” is meant for example any compound that is converted in vivo into a biologically active compound, for example, by passage through the stomach environment.

In some embodiments, a prebiotic composition may comprise an antioxidant to retard oxidation of one or more components. Additionally, the action of unwanted microorganisms can be controlled or prevented by preservatives such as antibacterial and antifungal agents, including, but not limited to parabens (e.g., methylparabens, propylparabens), chlorobutanol, phenol, sorbic acid, thimerosal or combinations thereof.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Therapeutic formulations useful in the invention may be prepared for storage by mixing an agent having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The dosages of prebiotic oligosaccharides, probiotics, and compositions described herein will depend on the specific clinical situation. Factors influencing actual dosage are, for example, the clinical scenario, for example the disease type and disease stage diagnosed in a subject, the age, weight, sex and overall health condition of a subject etc. As a general guideline, a prebiotic composition described herein may be administered within the range of 0.1-1000 mg(total active ingredient)/kg(body weight of subject)/day. In some embodiments, the prebiotic composition may be administered within the range of 1-300 mg/kg/day. In some embodiments, the prebiotic composition may be administered within the range of 5-20 mg/kg/day. In some embodiments, the prebiotic composition may be administered at a dosage of about 5 mg/kg/day. In some embodiments, the prebiotic composition may be administered at a dosage of about 10 mg/kg/day. In some embodiments, the prebiotic composition may be administered at a dosage of about 20 mg/kg/day.

The absolute amount of a prebiotic composition administered will depend upon a variety of factors including any concurrent treatment, the number of doses, the length of the treatment schedule, and the individual patient parameters including age, physical condition, health, size and weight. These are factors well known to those of ordinary skill in the art which can be assessed and used for determining an appropriate dosage or dosage range with no more than routine experimentation. In some embodiments, it is preferred that a maximum dose be used, that is, the highest safe, non-toxic dose according to sound medical judgment. In some embodiments, it is preferred to use the highest dose that is not associated with any or any severe side reactions in the subject. In some embodiments, it is preferred to use the minimal dose that provides a desired clinical result, for example, an acidification of an intestinal microenvironment, a decrease in the abundance of pathogenic microorganisms in the intestine, an increase in the abundance of beneficial microbiota in the intestine, and/or an amelioration of a symptom associated with an overabundance of pathogenic microorganisms in the intestine.

Multiple doses of prebiotic composition as described herein are also contemplated in some embodiments. In some instances, a prebiotic composition of the invention is administered with another medicament, for example, a medicament inhibiting the growth of pathogenic microorganisms in the intestine. In some such embodiments, a sub-therapeutic dosage of either the prebiotic composition or of the other medicament, or a sub-therapeutic dosage of both, is used in the treatment of a subject having, or at risk of developing a disease or disorder associated with the presence or an overabundance of pathogenic bacteria in the intestine. A “sub-therapeutic dose” as used herein refers to a dosage, which is less than that dosage which would produce a therapeutic result in the subject if administered in the absence of the other agent or agents. Thus, the sub-therapeutic dose of an agent is one which would not produce the desired therapeutic result in the subject in the absence of the administration of the agents of the invention. Therapeutic doses of many agents that are in clinical use are well known in the field of medicine, and additional therapeutic doses can be determined by those of skill without undue experimentation. Therapeutic dosages have been extensively described in references such as Remington's Pharmaceutical Sciences, 18th ed., 1990; as well as many other medical references relied upon by the medical profession as guidance for the treatment of diseases and disorders.

In some embodiments, a prebiotic composition provided herein is administered to a subject experiencing or suspected to experience a symptom related to an imbalance in the intestinal microbiota, for example, the presence or overabundance of pathogenic bacteria in the subject's intestine. In some embodiments, the active prebiotic and/or probiotic agents, or compositions, provided herein are administrated in an amount sufficient to prevent, reduce, or ameliorate at least one clinical symptom the subject is experiencing.

Sustained release strategies may also be employed in the methods described herein. Some such method use polymers to effect a sustained release of an agent from a composition. Both non-biodegradable and biodegradable polymeric matrices can be used to deliver a pro-biotic composition of the invention to the subject. Such polymers may be natural or synthetic polymers. The polymer is selected based on the period of time over which release is desired, generally in the order of a few hours. In some embodiments, a polymer is used that is broken down slowly in the intestine, thus releasing the prebiotic composition over time. The polymer optionally is in the form of a hydrogel that can absorb up to about 90% of its weight in water and further, optionally is cross-linked with multivalent ions or other polymers. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone. Examples of non-biodegradable polymers include ethylene vinyl acetate, poly(meth)acrylic acid, polyamides, copolymers and mixtures thereof. Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Several methods are disclosed herein of administering a subject with a prebiotic composition for the treatment of a disease or condition. It is to be understood that in each such aspect of the invention, the invention specifically includes, also, the prebiotic composition for use in the treatment or prevention of that disease or condition, as well as use of the compound for the manufacture of a medicament for the treatment or prevention of that disease or condition.

The route, frequency, dosage, and time frame of administration may vary depending on the condition identified in the subject. For example, a single administration of the agents and compositions described herein may be sufficient to reduce, prevent, or ameliorate an acute condition in the subject, whereas multiple doses, stretched out over a period of time, may be indicated in a subject experiencing a chronic disease or condition, such as chronic inflammatory bowel disease.

In some embodiments, administration may continue until a desired endpoint, e.g., a certain intestinal pH, or number or presence of beneficial microbiota, or absence or decreased abundance of pathogenic microorganisms in the intestine, or amelioration or reversal of a clinical symptom has been reached. In other embodiments, for example, in prophylactic embodiments, a prebiotic composition may be administered for a specified time and administration may be concluded without reaching a specific endpoint.

Without further elaboration, it is believed that one skilled in the art can, based on the above description, make and utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Materials and Methods

### In Vitro Fermentation

Fecal bacteria were cultured in carbohydrate-free basal medium according to Hughes' methods (Hughes S A, S. P., Gibson G R, McCleary B V, Rastall R A. 2008). This medium contained per liter: 2 g peptone, 2 g yeast extract, 0.1 g NaCl, 0.04 g K₂HPO₄, 0.01 g MgSO4.7H2O, 0.01 g CaCl2.6H2O, 2 g NaHCO₃, 0.005 g haemin (Sigma-Aldridge), 0.5 g L-cysteine HCl, 0.5 g bile salts, 2 mL Tween 80, 10 μl vitamin K, and 4 mL of 0.025% (w/v) resazurin solution. Anaerobic culture methods were those of Bryant (Bryant, M. P. 1972) using Hungate culture tubes, sealed with butyl rubber septa and maintained anaerobically using O₂-free CO₂.

Fresh fecal samples were collected from ten healthy babies, who had not received antibiotics or pre/probiotics for the previous 6 months and had no recent history of gastrointestinal disorder. The freshly obtained human faeces were homogenized in a blender for 60 s in phosphate-buffered sterile anaerobic saline solution (1:10 faeces to saline), and filtered through a double layer of sterile cheesecloth. To achieve a final concentration of 5 g/L test glycan in 1% faecal slurry, the glycan was partially dissolved in 9 mL medium for 1 h followed by addition of 1 mL of 10% faecal slurry. All tubes were incubated at 37° C. Culture fluid was taken for analysis after 48 h, a time that we had determined to be several hours into the maximum stationary phase for HMOS-treated and negative control populations. All experiments were carried out in triplicate. Samples were stored at −20° C. until completion of analyses.

A carbohydrate-free basal medium ZMB 1 was prepared according to Zhang et al. (Zhang G, M. D., Block D E. 2009) study the growth of bacteria strains in the presence of neutral oligosaccharides. Bacteria strains (Table 1) were obtained from the Japanese Collection of Microorganism (RIKEN BioResource Center, Japan), the American Type Culture Collection (Manassas, Va.). Reinforced clostridial medium (RCM) was used for growing Bifidobacteria and C. perfringens. LB was used for growing E. coli. Seed cultures of all bacteria were incubated overnight and the optical density at 600 nm reached 0.5, whereas 2 days of incubation was necessary for C. perfringens. All bacteria were grown in anaerobic conditions at 37° C., using an anaerobic chamber (DG250 Anaerobic Workstation, Don Whitley Scientific Limited, West Yorkshire, UK). 2′-FL (2 g/L) (Glycosyn, Inc. USA), 3-FL (2 g/L) (Glycosyn, Inc. USA), LDFT (1 g/L) (Glycosyn, Inc. USA), and their combination or HMOS were used as the sole carbon source. All the test materials were dissolved for an hour in the medium before inoculation with 10% (v/v) bacteria culture as above. ZMB1 containing the above test materials were inoculated with bacteria culture, serving as the treatment. ZMB1 containing no substrate was also inoculated with bacteria culture, to serve as control. ZMB1 containing FOS (2 g/L) was also inoculated with bacteria culture, to serve as positive control. All tubes were incubated at 37° C. Culture fluid was taken at 48 h. Growth was measured by optical density (OD=600 nm) in a microtiter plate.

### Determination of pH and Lactate Levels

Culture medium pH was recorded after 48 h of bacterial fermentation using a pH-meter (Corning, pH meter 240, USA). Lactate concentration in the medium was determined using a lactate assay kit (kit no. K607 -100; BioVision Inc., CA, USA). All experiments were carried out in triplicate.

### Analysis of Microbial Populations by Real-Time PCR

A fraction of the fermentation cultures (2 mL) were centrifuged at 12,000×g for 30 min. Then DNA was extracted from the cultures following the method of Zhu et al. (Zhu, W. Y., Williams, B. A., et al. 2003). Real-time PCR was used to determine the bacterial DNA present the end of fermentation culture. For this purpose, a series of genus-specific primer pairs were used according to Collado et al. (Collado, M. C., S. Delgado, A. Maldonado, and J. M. Rodríguez. 2009) (Table 1).

TABLE 1
Oligonucleotide primers used in this study

| Target | | Sequence (5′-3′) | Annealing | |
|---|---|---|---|---|
| bacterial species | Primers | Temp. | (° C.) | Reference |
| Bifidocacterium | g-Bifid-F | CTCCTGGAAACGGGTGG | 50 | (Matsuki,, |
| | | (SEQ ID NO: 1) | | et al., 2002) |
| | g-Bifid-R | GGTGTTCTTCCCGATATCTACA | | |
| | | (SEQ ID NO: 2) | | |
| C. perfringens | Clp-F | ATGCAAGTCGAGCGA(G/T)G | 55 | (Rinttilä, et |
| | | (SEQ ID NO: 3) | | al., 2004) |
| | Clp-R | TATGCGGTATTAATCT(C/T)CCTTT | | |
| | | (SEQ ID NO: 4) | | |
| E. coli | UidA784F | GTGTGATATCTACCCGCTTCG C | 56 | (Frahm, et |
| | | (SEQ ID NO: 5) | | al., 2003) |
| | UidA866R | AGAACGCTTTGTGGTTAATCAGGA | | |
| | | (SEQ ID NO: 6) | | |

PCR amplification and detection were performed using real-time PCR detection system (Bio-Rad Laboratories, Hercules, Calif., USA) according to the methods of Collado et al. (Collado, M. C., S. Delgado, A. Maldonado, and J. M. Rodríguez. 2009). Each reaction mixture (25 μL) was composed of iQ™ SYBR [symbol] Green Supermix (Bio-Rad Laboratories), 1 μL of each of the specific primers at a concentration of 0.25 μM and 1 μL of template DNA. The fluorescent products were detected at the last step of each cycle. A melting curve analysis was made after amplification to distinguish the targeted PCR product from the non-targeted PCR product. Standard curves were eight 10-fold dilutions of bacterial DNA extracted from pure cultures of between 2 to 9 log₁₀ colony forming units (CFUs) of each of the following selected representative species: Bifidobacterium infantis S12 ATCC 15697, Clostridum perfringens ATCC13124, Escherichia coli H10407 ATCC 35401.

### Determination of the Consumption of Each Oligosaccharide

Samples were thawed and centrifuged at 4000×g for 15 minutes at 4° C. The clear supernatant (0.5 mL) was treated with 0.25 mL of a fresh aqueous solution of sodium borohydride (0.5 M). After vigorous mixing, the reduction mixture was kept overnight at 4° C. then treated with 0.25 mL acetic acid (0.5 M). In a Serological pipette (5×0.5 cm), from the lower, glass wool, sand, treated 0.6 g (3 meq) AG50W-X8 cation-exchange resin (BioRad), 0.9 g (3 meq) AG1-X8 anion-exchange resin (acetate form, BioRad, Hercules Calif.), celite were packed. The AG50W-X8 cation-exchange resin (hydrogen form, BioRad, Hercules Calif.) was treated by 1 M pyridine, 1 h×3 times, resulted in pyridinium form. The above column was activated with 0.5 mL water, followed by the reduced samples (1 mL) applied to the column. The sample tube was rinsed with water (0.5 mL), and the resin column washed with an additional 18 mL of water. The eluates were neutral oligosaccharides, followed by frozen with dry ice in ethanol before lyophilization. The neutral oligosaccharides in samples were analyzed by an Agilent HPLC (1200 series) with a triple-quadrupole mass spectrometer (6460), equipped with a porous graphite column (3 μm, 100×2.1 mm, Hypercarb, Thermo Scientific, Waltham, Mass.) set for 25° C. Methods were validated by authentic oligosaccharides from GlycoSeparations (Moscow, Russia) (Newburg, D. S. 2001).

### Statistical Analysis

Data are expressed as mean±SEM. The statistical significance of differences between groups was determined by one-way ANOVA. When differences were found, Student's t-test was used for pairwise comparisons; P≦0.05 was considered significant.

### Results

### pH and Lactate Variation in the Fermentation Culture

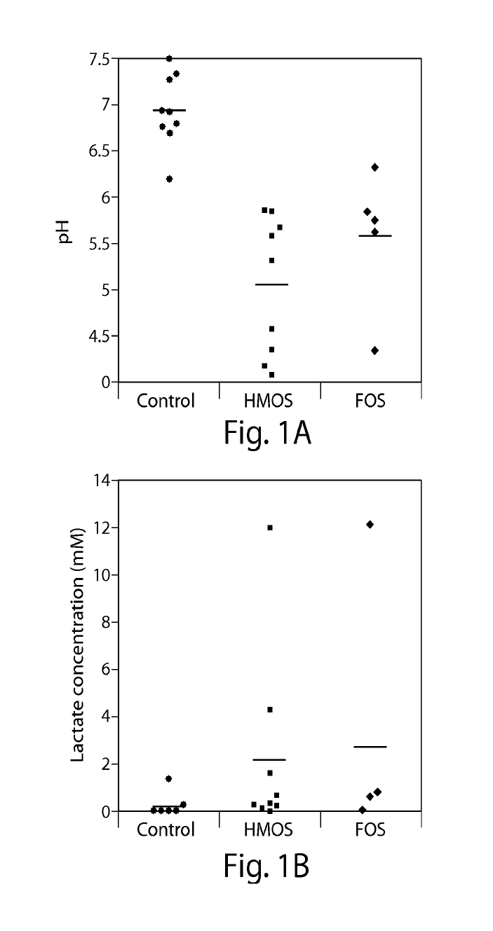

Human milk glycans affects colonization directly, by selecting for bacteria to use this unique glycans, and indirectly, when fermented to lactate and short chain fatty acid, making the gut acidic and stimulating the growth of some bacteria while inhibiting colonization by many pathogenic organisms. The pH and lactate in the fermentation cultures were detected to determine if the HMOS was fermented by the faecal microbiota. FIG. 1 showed that the pH in the groups supplemented with HMOS were significantly lower than that in unsupplemented control and positive control supplemented with FOS (P<0.05). And the lactate concentrations in the HMOS supplemented groups were significantly higher than that in control (P<0.05).

### Bacterial Population Changes

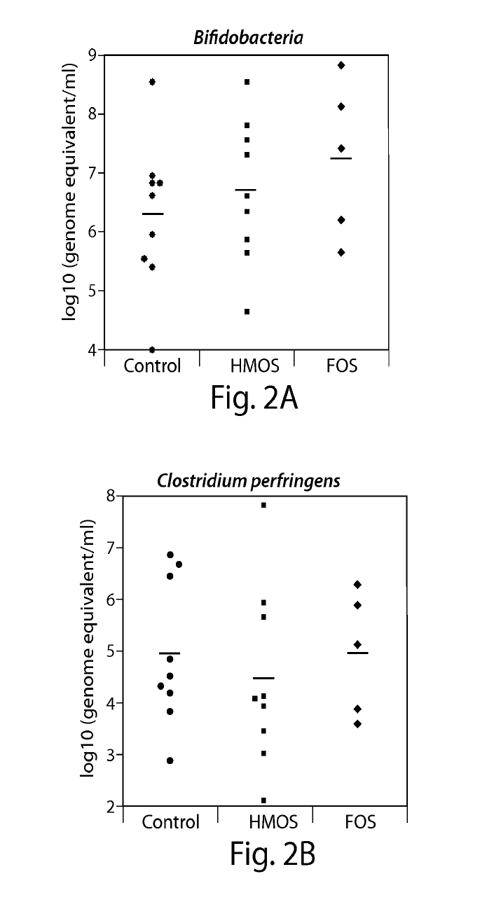

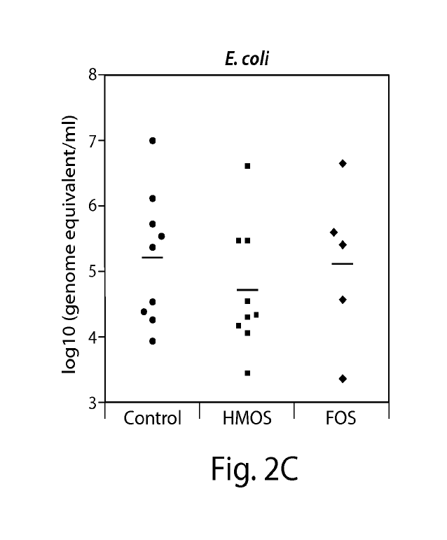

To determine if the presence of HMOS can alter the distribution of the gut microbiota, the relative amounts of microorganisms were determined in the presence of HMOS and FOS. Relative amounts of bacterial species in the donor faecal samples were characterized by real-time PCR in the presence of HMOS and FOS. FIG. 2 showed that HMOS increased the number of Bifidobacteria, while the numbers of E. coli and C. perfringens declined.

### Consumption of Each Oligosaccharide

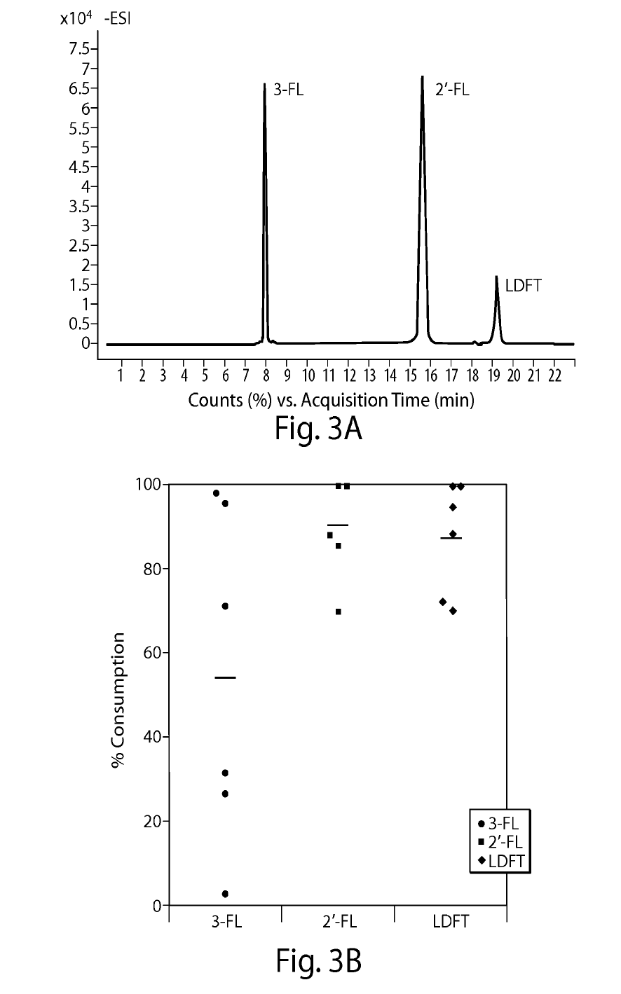

To determine the specific HMOS structures consumed by the faecal bacterial species, supernatants from the faecal culture were recovered after fermentation, and remaining HMOS were purified, reduced, and profiled by LC-MASS as previously described by Newburg (Newburg, D. S. 2001). The major neutral oligosaccharides (including 2′-FL, 3-FL and LDFT) were monitored. FIG. 3. The average consumption of 2′-FL and LDFT for all donor faecal microbiota were more than 90%, while for 3-FL, the consumption is about 53%. These data indicate that 2′-FL and LDFT metabolized by faecal bacteria while 3-FL appears to be more resistant to catabolism by these organisms as it was found to be catabolized by an organism that is only present in small numbers.

### Effect of HMOS and Oligosaccharide Combinations on Growth of Different Bacterial Strains

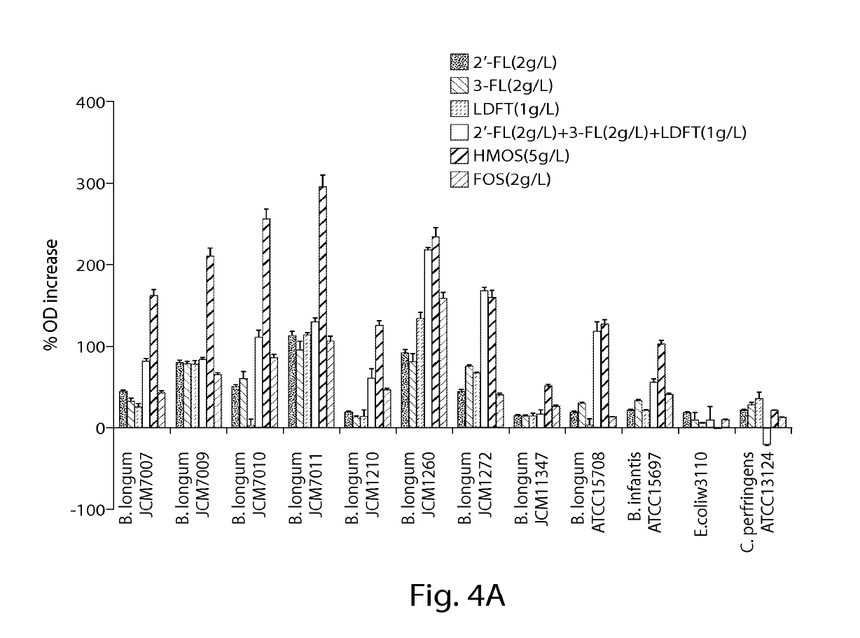

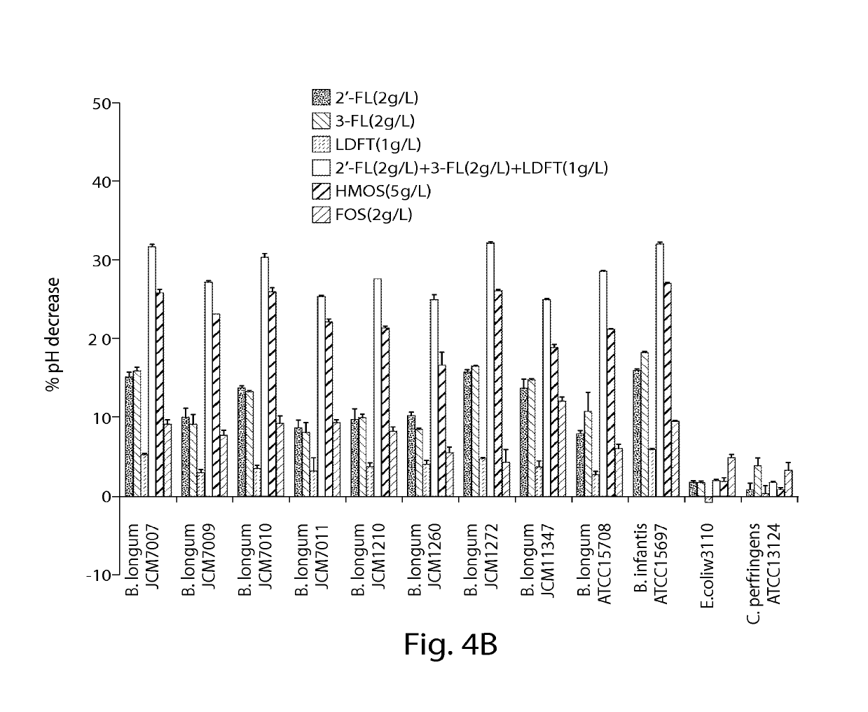

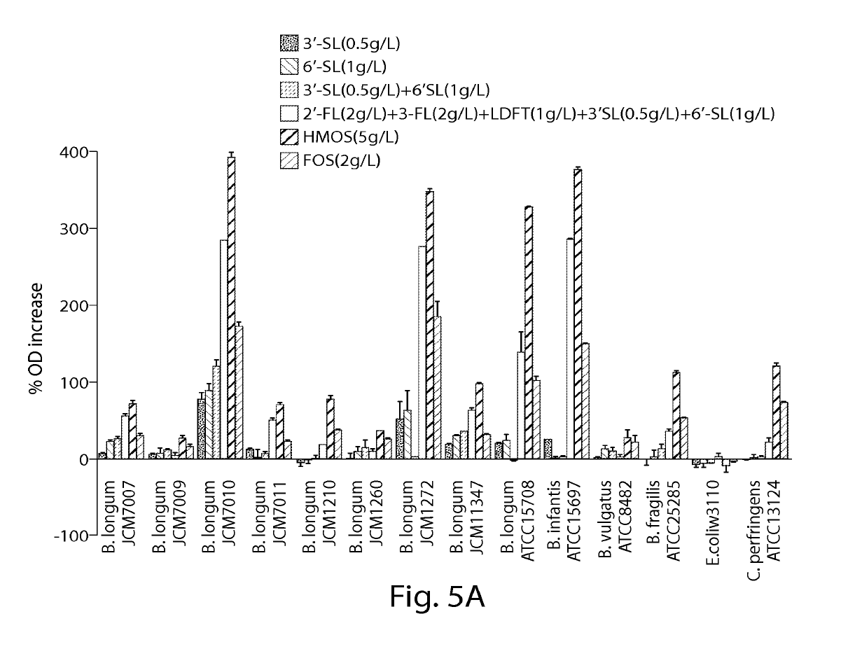

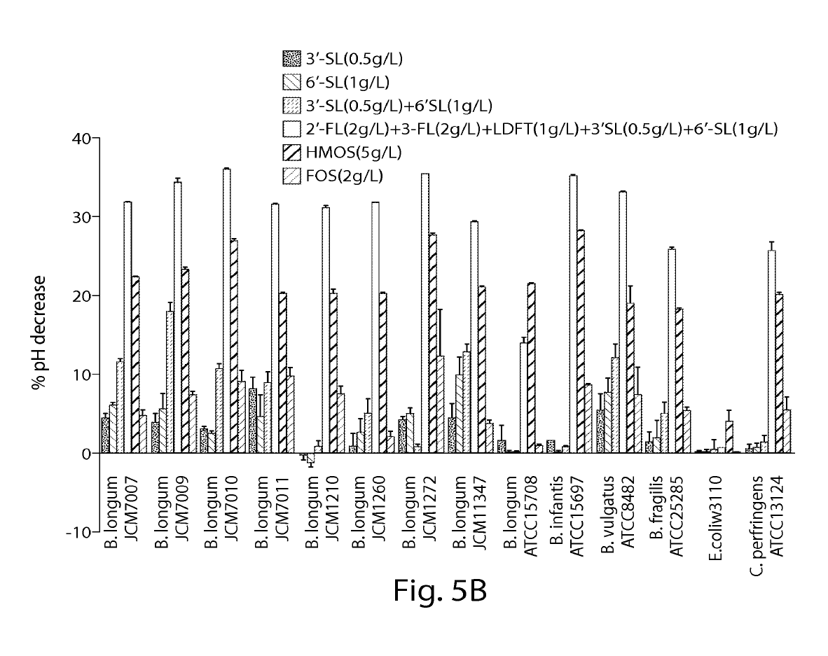

Ten different Bifidobacteria used in this study (see Table 2 below) could utilize 2′-FL, 3-FL, LDFT, a combination thereof, 3′-SL, 6′-SL, a combination of 3′-SL and 6′-SL, and a combination of all these fucosylated oligosaccharides and siallyllactoses as a primary carbon source at concentrations indicated in FIGS. 4 and 5.

FIG. 4 showed that all the fucosylated oligosaccharides, 2′-FL, 3-FL, LDFT, and HMOS could significantly increase the growth of the Bifidobacteria sp. and decrease the pH in the culture. For E. coli and C. perfringens, there is no significant increase on its growth and decrease in pH. Combination of the three individual fucosylated oligosaccharides have a cumulative effect on the growth increased and pH decreased accordingly. And the effect is equivalent to total HMOS. These data suggest that the neutral oligosaccharides can be used as a carbon source by these prebiotic organisms and inhibit the growth of pathogenic organisms.

TABLE 2
Bacterial Strains used in this study

| Species | Origin |
|---|---|
| Bifidobacterium longum JCM 7007 | Faeces of human infant |
| Bifidobacterium longum JCM 7009 | Faeces of human infant |
| Bifidobacterium longum JCM 7010 | Faeces of human infant |
| Bifidobacterium longum JCM 7011 | Faeces of human infant |
| Bifidobacterium longum JCM 1210 | Faeces of human infant |
| Bifidobacterium longum JCM 1260 | Faeces of human infant |
| Bifidobacterium longum JCM 1272 | Faeces of human infant |
| Bifidobacterium longum JCM 11347 | Faeces of human infant |
| Bifidobacterium longum ATCC 15708 | Faeces of human infant |
| Bifidobacterium infantis ATCC 15697 | Faeces of human infant |
| Clostridium perfringens ATCC 13124 | n/a |
| Escherichia coli W3110 | Human faeces |

In sum, the combinations of fucosylated neutral oligosaccharides showed greater activity in promoting the growth of Bifidobacteria and decreasing the pH in the microenvironment of the bacteria than the individual ones. As shown in FIG. 4, the combination of the three tested fucosylated oligosaccharides showed equal or greater activity in promoting the growth and/or decreasing pH of certain Bifidobacteria strains as compared to the prebiotic effect of the total oligosaccharides from human milk on a weight basis. These oligosaccharides did not stimulate E coli and C perfringens, which are not mutualist with humans.

Sialyl oligosaccharides, in combination with other pure oligosaccharides, showed similar activity. Sialyllactoses such as 3′-SL and 6′-SL alone showed prebiotic effects on some Bifidobacteria strains. FIG. 5. Surprisingly, the combinations of these two sialyllactoses with fucosylated oligosaccharides (2′-FL, 3-FL, and LDFT) showed activities in growth-promoting and pH-decreasing at levels equal to or greater than those of total natural HMOS mixture. The pH-decreasing activity was especially pronounced in the mixtures containing the sialyllactoses; it greatly exceeded that of the natural HMOS mixture. FIG. 5.

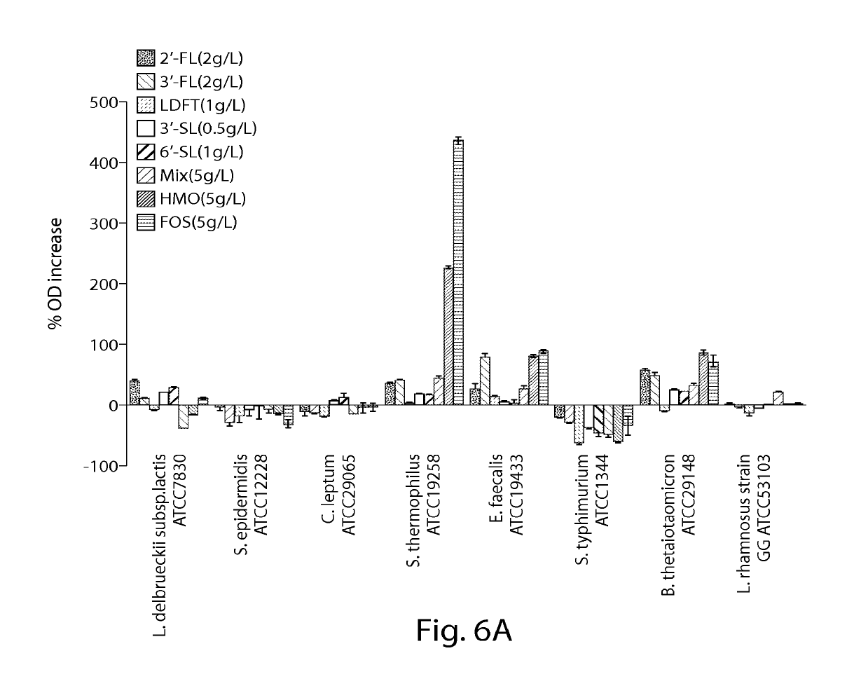

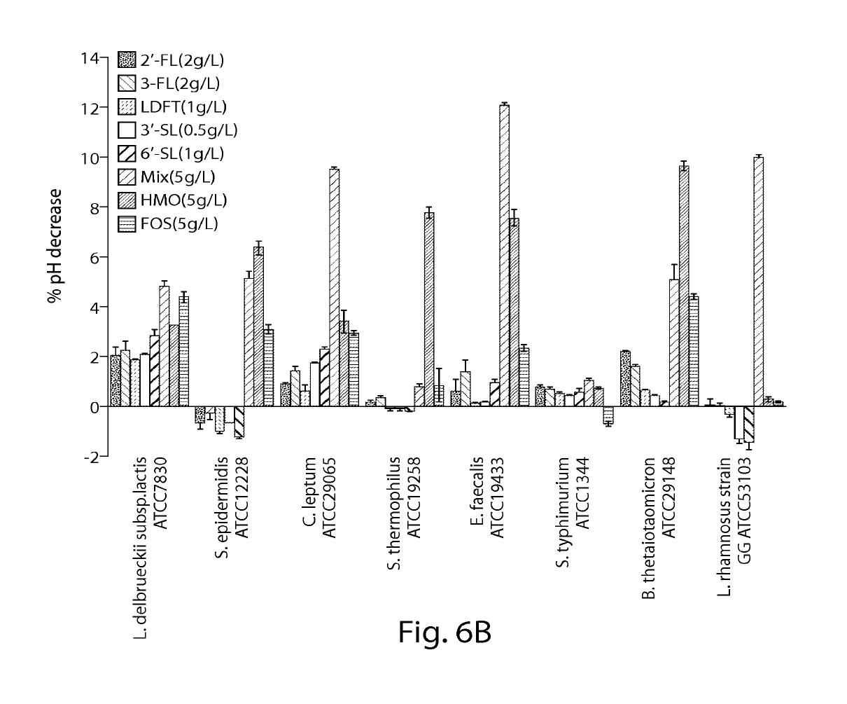

The effects of individual oligosaccharides and their mixtures on panel of pathogens and other denizens of the microbiota were also tested. FIG. 6. FOS (5 g/L) was used as a positive control. The results thus obtained indicate that some of the tested bacterial strains do not utilize these oligosaccharides, some use FOS well, but many use HMOS and the mixture of the oligosaccharides. FIG. 6. The results also show the effects of the mixture on certain bacterial strains are higher than those of the HMOS on the same strains.

Overall, the studies described above showed that through in vitro fermentation, the numbers of bifidobacteria supplemented with HMOS, fucosylated oligosaccharides (2′-FL, 3-FL, LDFT, or a mixture thereof), siallyllactose (3′-SL, 6′-SL, or a mixture thereof), and a combination of fucosylated oligosaccharide and siallyllactose, were significantly higher than that in control. At the same time, all tested oligosaccharide combinations and HMOS could significantly stimulate growth and decrease the pH in all the ten culture of Bifidobacteria. These results suggest that the prebiotic, bifidogenic effects of HMOS are structure-specific and may vary depending on the HMOS composition in the milk of different individuals or change over the course of lactation. Increased colonization by bifidobacteria in breast-fed infants may enhance subsequent long-term formation of a stable microbial ecosystem by favouring symbiotic anaerobes and inhibiting colonization by enteric pathogens, protecting the infant from disease.

### REFERENCES

Backhed F, Manchester J K, Semenkovich C F, Gordon J I. 2007. Mechanisms underlying the resistance to diet-induced obesity in germ-free mice. Proc Natl Acad Sci USA, 104:979-984.

Bryant M P. 1972. Commentary on the Hungate technique for culture of anaerobic bacteria. Am J Clin Nutr, 25:1324-1328.

Bullock N R, Booth J C, Gibson G R. 2004. Comparative composition of bacteria in the human intestinal microflora during remission and active ulcerative colitis. Curr Issues Intest Microbiol, 5:59-64.

Collado M C, S. Delgado, A. Maldonado, and J. M. Rodríguez. 2009. Assessment of the bacterial diversity of breast milk of healthy women by quantitative real-time PCR. Lett Appl Microbiol, 48:523-528.

Frahm E, and U. Obst. 2003. Application of the fluorogenic probe technique (TaqMan PCR) to the detection of Enterococcus spp. and Escherichia coli in water samples. J Microbiol Methods, 52:123-131.

German J B, Freeman S L, Lebrilla C B, Mills D A. 2008. Human milk oligosaccharides: evolution, structures and bioselectivity as substrates for intestinal bacteria. Nestle Nutr Workshop Ser Pediatr Program, 62:205-218; discussion 218-222.

Greenstein R J. 2003. Is Crohn's disease caused by a mycobacterium? Comparisons with leprosy, tuberculosis, and Johne's disease. Lancet Infect Dis, 3:507-514.

Gyorgy P, R. F. Norris, and C. S. Rose. 1954. Bifidus factor. I. A variant of Lactobacillus bifidus requiring a special growth factor. Arch Biochem Biophys, 48:193-201.

Hamosh M, Goldman A S. 1986. Human lactation 2, Maternal and Environmental Factors. New York (NY): Plenum Press.

Heavey P M, Rowland I R. 2004. Microbial-gut interactions in health and disease. Gastrointestinal cancer. Best Pract Res Clin Gastroenterol, 18:323-336.

Heijnen A M, Brink E J, Lemmens A G, Beynen A C. 1993. Ileal pH and apparent absorption of magnesium in rats fed on diets containing either lactose or lactulose. Br J Nutr, 70:747-756.

Huang P, Farkas T, Marionneau S, Zhong W, Ruvoen-Clouet N, Morrow A L, Altaye M, Pickering L K, Newburg D S, LePendu J, et al. 2003. Noroviruses bind to human ABO, Lewis, and secretor histo-blood group antigens: identification of 4 distinct strain-specific patterns. J Infect Dis, 188:19-31.

Hughes S A S P, Gibson G R, McCleary B V, Rastall R A. 2008. In vitro fermentation of oat and barley derived beta-glucans by human faecal microbiota. FEMS Microbiol Ecol., 64:482-493.

Ley R E, Backhed F, Turnbaugh P, Lozupone C A, Knight R D, Gordon J I. 2005. Obesity alters gut microbial ecology. Proc Natl Acad Sci USA, 102:11070-11075.

Ley R E, Turnbaugh P J, Klein S, Gordon J I. 2006. Microbial ecology: human gut microbes associated with obesity. Nature, 444:1022-1023.

LoCascio R G, Desai P, Sela D A, Weimer B, Mills D A. 2010. Broad conservation of milk utilization genes in Bifidobacterium longum subsp. infantis as revealed by comparative genomic hybridization. Applied and environmental microbiology, 76:7373-7381.

LoCascio R G, Ninonuevo M R, Freeman S L, Sela D A, Grimm R, Lebrilla C B, Mills D A, German J B. 2007. Glycoprofiling of bifidobacterial consumption of human milk oligosaccharides demonstrates strain specific, preferential consumption of small chain glycans secreted in early human lactation. J Agric Food Chem, 55:8914-8919.

Macfarlane G T, Macfarlane S. 1997. Human colonic microbiota: ecology, physiology and metabolic potential of intestinal bacteria. Scand J Gastroenterol Suppl, 222:3-9.

Marcobal A, Barboza M, Froehlich J W, Block D E, German J B, Lebrilla C B, Mills D A. 2010. Consumption of human milk oligosaccharides by gut-related microbes. Journal of agricultural and food chemistry, 58:5334-5340.

Marionneau S, Ruvoen N, Le Moullac-Vaidye B, Clement M, Cailleau-Thomas A, Ruiz-Palacois G, Huang P, Jiang X, Le Pendu J. 2002. Norwalk virus binds to histo-blood group antigens present on gastroduodenal epithelial cells of secretor individuals. Gastroenterology, 122:1967-1977.

Matsuki T, K. Watanabe, J. Fujimoto, Y. Miyamoto, T. Takada, K. Matsumoto, H. Oyaizu, and R. Tanaka. 2002. Development of 16S rRNA-gene-targeted group-specific primers for the detection and identification of predominant bacteria in human feces. Appl Environ Microbiol, 68:5445-5451.

Morrow A L, Meinzen-Derr J, Huang P, Schibler K R, Cahill T, Keddache M, Kallapur S G, Newburg D S, Tabangin M, Warner B B, et al. 2011. Fucosyltransferase 2 non-secretor and low secretor status predicts severe outcomes in premature infants. The Journal of pediatrics, 158:745-751.

Nanthakumar N N, Dai D, Meng D, Chaudry N, Newburg D S, Walker W A. 2005. Regulation of intestinal ontogeny: effect of glucocorticoids and luminal microbes on galactosyltransferase and trehalase induction in mice. Glycobiology, 15:221-232.

Newburg D S. 1996. Oligosaccharides and glycoconjugates in human milk: their role in host defense. J Mammary Gland Biol Neoplasia, 1:271-283.

Newburg D S. 1997. Do the binding properties of oligosaccharides in milk protect human infants from gastrointestinal bacteria? The Journal of nutrition, 127:980S-984S.

Newburg D S. 2000. Oligosaccharides in human milk and bacterial colonization. J Pediatr Gastr Nutr, 30 Suppl 2:S8-17.

Newburg D S. 2001. Bioactive components of human milk Kluwer Academic/Plenum Publishers:Waltham.

Newburg D S, Pickering L K, McCluer R H, Cleary T G. 1990a. Fucosylated oligosaccharides of human milk protect suckling mice from heat-stabile enterotoxin of Escherichia coli. J Infect Dis, 162:1075-1080.

Newburg D S, Pickering L K, McCluer R H, Cleary T G. 1990b. Fucosylated oligosaccharides of human milk protect suckling mice from heat-stabile enterotoxin of Escherichia coli. The Journal of infectious diseases, 162:1075-1080.

Newburg D S, Ruiz-Palacios G M, Altaye M, Chaturvedi P, Meinzen-Derr J, Guerrero Mde L, Morrow A L. 2004. Innate protection conferred by fucosylated oligosaccharides of human milk against diarrhea in breastfed infants. Glycobiology, 14:253-263.

Newburg D S, Ruiz-Palacios G M, Morrow A L. 2005. Human milk glycans protect infants against enteric pathogens. Annual review of nutrition, 25:37-58.

Ohta A, Ohtsuki M, Uehara M, Hosono A, Hirayama M, Adachi T, Hara H. 1998. Dietary fructooligosaccharides prevent postgastrectomy anemia and osteopenia in rats. The Journal of nutrition, 128:485-490.

Rinttila T, A. Kassinen, E. Malinen, L. Krogius, and A. Palva. 2004. Development of an extensive set of 16S rDNA-targeted primers for quantification of pathogenic and indigenous bacteria in faecal samples by real-time PCR. J Appl Microbiol, 97:1166-1177.

Ruiz-Palacios G M, Cervantes L E, Ramos P, Chavez-Munguia B, Newburg D S. 2003. Campylobacter jejuni binds intestinal H(O) antigen (Fuc alpha 1, 2Ga1 beta 1, 4GlcNAc), and fucosyloligosaccharides of human milk inhibit its binding and infection. The Journal of biological chemistry, 278:14112-14120.

Videla S, Vilaseca J, Guarner F, Salas A, Treserra F, Crespo E, Antolin M, Malagelada J R. 1994. Role of intestinal microflora in chronic inflammation and ulceration of the rat colon. Gut, 35:1090-1097.

Ward R E, M. Ninonuevo, D. A. Mills, C. B. Lebrilla, and J. Bruce German. 2006. In vitro fermentation of breast milk oligosaccharides by Bifidobacterium infantis and Lactobacillus gasseri. Appl Environ Microbiol, 72:4497-4499.

Ward R E, Ninonuevo M, Mills D A, Lebrilla C B, German J B. 2006. In vitro fermentation of breast milk oligosaccharides by Bifidobacterium infantis and Lactobacillus gasseri. Applied and environmental microbiology, 72:4497-4499.

Yolken R H, Peterson J A, Vonderfecht S L, Fouts E T, Midthun K, Newburg D S. 1992. Human milk mucin inhibits rotavirus replication and prevents experimental gastroenteritis. The Journal of clinical investigation, 90:1984-1991.

Zhang G M D, Block D E. 2009. Development of chemically defined media supporting high-cell-density growth of lactococci, enterococci, and streptococci. Appl Environ Microbiol., 75:1080-1087.

Zhu W Y, Williams B A, Konstantinov S R, Tamminga S, De Vos W M, Akkermans A D. 2003. Analysis of 16S rDNA reveals bacterial shift during in vitro fermentation of fermentable carbohydrate using piglet faeces as inoculum. Anaerobe, 9:175-180.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A prebiotic composition consisting essentially of a sialyllactose and a fucosylated oligosaccharide, wherein the sialyllactose is 3′-sialyllactose (3′-SL), 6′-sialyllactose (6′-SL), or a mixture thereof, and wherein the fucosylated oligosaccharide comprises an α1,2-fucosyl, an α1,3-fucosyl, and/or an α1,4-fucosyl residue.

2. The prebiotic composition of claim 1, wherein the sialyllactose is a mixture of 3′-SL and 6′-SL.

3. The prebiotic composition of claim 1, wherein the fucosylated oligosaccharide comprises a fucosylated neutral oligosaccharide.

4. The prebiotic composition of claim 1, wherein the fucosylated oligosaccharide is 2′-fucosyllactose (2-FL), 3-fucosyllactose (3-FL), lactodifucotetraose (LDFT), or a mixture thereof.

5. The prebiotic composition of claim 1, wherein the fucosylated oligosaccharide is a combination of:
2′-FL and 3-FL;
2′-FL and LDFT;
3-FL and LDFT; or
2′-FL, 3-FL, and LDFT.

6. The prebiotic composition of claim 1, wherein the composition consists essentially of a mixture of 3′-SL, 6′-SL, 2′-FL, 3-FL, and LDFT.

7. The prebiotic composition of claim 1, wherein the composition further contains a probiotic.

8. The prebiotic composition of claim 7, wherein the probiotic is a population of bifidobacteria, lactobacilli, Bacteriodes fragilis, Bacteriodes thetaiotaomicron, Enterococcus faecalis, Staphylococcus epidermides, Enterobacter aerogenes, Enterobacter cloacae, or a combination thereof.

9. The prebiotic composition of claim 8, wherein the population of bifidobacteria is B. longum, B. infantis, or a mixture thereof.

10. The prebiotic composition of claim 9, wherein the population of bifidobacteria is B. longum JCM7007, JCM7009, JCM7010, JCM7011, JCM1210, JCM1260, JCM1272, JCM11347, ATCC15708, B. infantis ATCC15697, or a mixture thereof.

11. The prebiotic composition of claim 1, wherein the composition comprises 3′-SL and 6′-SL at a ratio ranging from 4:1 to 1:2.

12. A method of increasing the proliferation of bifidobacteria, the method comprising contacting a population of bifidobacteria with a prebiotic composition comprising a sialyllactose and a fucosylated oligosaccharide in an amount effective in increasing the proliferation of the bifidobacteria population.

13. The method of claim 12, wherein the sialyllactose is 3′-sialyllactose (3′-SL), 6′-sialyllactose (6′-SL), or a mixture thereof.

14. The method of claim 11, wherein the fucosylated oligosaccharide comprises an α1,2-fucosyl, an α1,3-fucosyl, and/or an α1,4-fucosyl residue.

15. The method of claim 12, wherein the fucosylated oligosaccharide comprises a fucosylated neutral oligosaccharide.

16. The method of claim 12, wherein the fucosylated oligosaccharide is 2′-fucosyllactose (2-FL), 3-fucosyllactose (3-FL), lactodifucotetraose (LDFT), or a mixture thereof.

17. The method of claim 12, wherein the contacting step is performed in vitro.

18. The method of claim 12, wherein the contacting step comprises administering the prebiotic composition to a subject in need thereof.

19. The method of claim 18, wherein the prebiotic composition is administered orally to the subject.

20. The method of claim 18, wherein the subject is a human.

21. The method of claim 18, wherein the subject is an infant.

22. The method of claim 21, wherein the infant is a neonatal infant.

23. The method of claim 18, wherein the subject is a subject suffering from, suspected of having, or at risk for a disease associated with an underrepresentation of beneficial microorganisms or the presence or overabundance of pathogenic bacteria in the intestine.

24. The method of claim 18, wherein the subject is suffering from, suspected of having, or at risk for irritable bowel syndrome or inflammatory bowel disease.

25. The method of claim 18, wherein the prebiotic composition is administered to the subject in an amount effective to decrease the pH in the microenvironment of the bifidobacteria.

26. The method of claim 18, wherein the prebiotic composition is administered to the subject in an amount effective to decrease the proliferation rate of a pathogenic bacterium.

27. The method of claim 12, wherein the population of bifidobacteria comprises Bifidobacterium longum, Bifidobacterium infantis, or a mixture thereof.

28. The method of claim 27, wherein the pathogenic bacterium is Escherichia coli or Clostridium perfringens.

29. The method of claim 12, wherein the prebiotic composition further comprises a probiotic.

30. The method of claim 29, wherein the probiotic is a population of bifidobacteria, lactobacilli, Bacteriodes fragilis, Bacteriodes thetaiotaomicron, Enterococcus faecalis, Staphylococcus epidermides, Enterobacter aerogenes, Enterobacter cloacae, or a combination thereof.

31. The method of claim 30, wherein the population of bifidobacteria is B. longum, B. infantis, or a mixture thereof.

32. The method of claim 31, wherein the population of bifidobacteria is B. longum JCM7007, JCM7009, JCM7010, JCM7011, JCM1210, JCM1260, JCM1272, JCM11347, ATCC15708, B. infantis ATCC15697, or a mixture thereof.

33. The method of claim 12, wherein the prebiotic composition comprises 3′-SL and 6′-SL at a ratio ranging from 4:1 to 1:2.

34-37. (canceled)
